(19) United States
(12) Patent Application Publication
(10) Pub. No.: US 2024/0139113 A1
(43) Pub. Date: May 2, 2024

(54) LIPID NANOPARTICLE WITH TARGET INTEGRIN FUNCTION AND USES THEREOF

(71) Applicant: Hung-Yun Lin, Taipei City (TW)

(72) Inventor: Hung-Yun Lin, Taipei City (TW)

(21) Appl. No.: 17/975,968

(22) Filed: Oct. 28, 2022

(51) Int. Cl. A61K 9/51; A61K 9/00; A61K 9/127; A61K 31/704; A61K 47/28

(52) U.S. Cl. CPC A61K 9/5123; A61K 9/0019; A61K 9/1271; A61K 31/704; A61K 47/28

(57) ABSTRACT

The present disclosure provides a lipid nanoparticle and uses of the lipid nanoparticle for treating cancer, targeting integrin αvβ3, anti-angiogenesis to prevent cancer metastasis and reducing the cytotoxicity of anti-cancer drugs to normal cells. In addition, since it can inhibit angiogenesis, it also can be used in vascular related diseases. The lipid nanoparticle provides targeting cell surface integrin αvβ3, the nano-liposome specific against digestion system-related cancers locally and systemically. The lipid nanoparticle of the present disclosure would target cancer cells instead of normal cells. Even normal cells contain integrin αvβ3, however, the lipid nanoparticle of the present disclosure only recognizes the conformation of integrin αvβ3 on the cancer cells. The lipid nanoparticle of the present disclosure can equip with other targeting molecules, payload with other anti-cancer drugs, and can combine with radiation therapy and reduce radiation therapeutic threshold.

### BACKGROUND OF THE INVENTION

### 1. Field of the Invention

The present invention relates to a lipid nanoparticle with target integrin αvβ3 function and uses thereof.

### 2. The Prior Art

Cancers are the biggest killer of human disease-related mortality. Although digestion system-related cancer may not be the number one killer in cancer-induced mortality, the death ratio is still huge and cannot be ignorant. Several types of digestion system-related cancer such as cholangiocarcinomas, malignant tumors of the biliary tract, and pancreatic cancer are not easy to detect in the early stage. It is epidemiologically important throughout the world and effective chemotherapies for some types of tumors are not yet available.

Except for some types of cancers, digestion system-related cancers have universally poor outcomes, with surgical resection offering the only chance for cure. They are associated with a high mortality rate because of their difficulty in early detection. In addition, they are resistant to most chemotherapeutic agents. Cisplatin or gemcitabine has been used as a standard chemotherapeutic agent on cholangiocarcinoma (CCA), however, recent studies have found that several gemcitabine-resistant cell lines are cross-resistant to 5-fluorouracil (5-FU), doxorubicin, and paclitaxel indicating their multidrug-resistant nature. On the other hand, gefitinib has been reported to be a radiosensitizer, which inhibits radiation-induced phosphorylation of epidermal growth factor receptor (EGFR) and the downstream pathway and therefore enhances radiosensitivity in CCA cells and other cancer cells.

Integrin αvβ3 is a structural protein of the plasma membrane that is important to extracellular matrix protein-cell interactions. Studies indicate that integrin αvβ3 may be an excellent target for cancer chemotherapy. However, there is no effective medical agent available.

Recently, studies have shown that thyroid hormone (T₃ and T₄), thyroxine can stimulate cancer proliferation via its specific binding to cell surface integrin αvβ3 to activate signal transduction pathways. Thyroxine-integrin αvβ3 axis has been shown to play an important role in cancer progression in several types of cancers.

Thyroid hormones influence angiogenic signaling in mesenchymal stem cells (MSCs) via integrin αvβ3 and further substantiate the anti-angiogenic activity of thyroid hormone deaminated analog, tetraiodothyroacetic acid (Tetrac) in the tumor microenvironment.

Checkpoint programmed cell death protein 1 (PD-1)/programmed death-ligand 1 (PD-L1) plays an important role in cancer proliferation which also protects tumor cells against immune demolition. Increased expression of PD-L1 has been shown to positively correlate with cancer progression. Recently researchers discovered that the thyroid hormone, thyroxine, is able to induce PD-L1 expression and protein accumulation in breast cancer cells and oral cancer cells.

In the setting of cancer, the growth factor may contribute to metastasis via actions on angiogenesis, lymphangiogenesis, and transendothelial migration of prometastatic cancer cells. Signals of epidermal growth factor (EGF), insulin-like growth factor-1 (IGF-1), and transforming growth factor-β (TGF-β) cross-talk with integrin αvβ3 to stimulate cancer cell proliferation. TGF-β regulates cell proliferation, differentiation, and functional behavior. Thyroid hormone potentiates TGF-β-induced normal airway smooth muscle cell proliferation, and this potentiation is mediated by the hormone receptor on integrin αvβ3. Integrin αvβ3 cross-links with growth factor-induced signal transduction pathways. 15% of the EGFR gene mutations in the kinase domain are in biliary cancer. K-ras mutation and aberrant expression of p53 are present in one-third of the intrahepatic CCAs.

Carcinoembryonic antigen-related cell adhesion molecule 6 (CEACAM6) plays a crucial role in the tumorigenesis of cancer. CEACAM6 promotes tumor angiogenesis and vasculogenic mimicry formation via focal adhesion kinase (FAK) signaling in gastric cancer. Decreasing phosphorylation of FAK and paxillin also significantly reduces gastric cancer metastasis via FAK signaling. Blocking CEACAM6 function by a specific antibody is also shown to reduce cancer growth. On the other hand, FAK is downstream of integrin αvβ3. Therefore, integrin αvβ3 can crosstalk with CEACAM6 directly or indirectly through FAK signaling. Other signals such as phosphoinositide 3-kinase (PI3K) activation may also play roles in the crosstalk between αvβ3 and CEACAM6. Integrin αvβ3 cross-talks with CEACAM6.

The synergism of T₄ and TGF-β is blocked by thyroid hormone analogue, Tetrac. The contributions of dysregulated TGF-β pathway to the processes of oncogenic transformation and metastasis are also now widely appreciated and have recently been reviewed. Expression of the EGFR gene by tumor cells is associated with drug resistance, metastasis, and angiogenic support of metastases. Thyroid hormone induces transcription of EGFR. Because of its association with drug resistance and metastasis, EGFR protein is an established chemotherapeutic target.

In view of the shortcomings of the current treatment of cancer and target drugs, there are still side effects, cytotoxicity to normal cells and poor effects. In order to solve the above-mentioned problems, those skilled in the art urgently need to develop a novel and effective pharmaceutical composition for the benefit of a large group of people in need thereof.

### SUMMARY OF THE INVENTION

An objective of the present invention is to provide a lipid nanoparticle, comprising a hollow lipid sphere and tetraiodothyroacetic acid (Tetrac) covalently linked to the hollow lipid sphere, wherein the hollow lipid sphere is composed of a lipid, and the lipid comprises a hydrogenated soy phosphatidylcholine (HSPC), a cholesterol and 1,2-distearoyl-sn-glycero-3-phosphoethanolamine-polyethylene glycol (DSPE-PEG), wherein the DSPE-PEG is covalently linked to the Tetrac.

According to an embodiment of the present invention, the lipid nanoparticle further comprises a first drug, wherein the first drug is encapsulated in the hollow lipid sphere.

According to an embodiment of the present invention, the first drug is doxorubicin.

According to an embodiment of the present invention, the lipid nanoparticle further comprises a second drug, wherein the second drug is an anti-cancer drug.

According to another embodiment of the present invention, the lipid nanoparticle can also inhibit the expression of carcinoembryonic antigen-related cell adhesion molecule 6 (CEACAM6).

Another objective of the present invention is to provide a method for treating cancer, comprising administering to a subject in need thereof a pharmaceutical composition comprising an effective amount of the abovementioned lipid nanoparticle.

According to an embodiment of the present invention, the cancer is colorectal cancer, bile duct cancer, pancreatic cancer, breast cancer or lung cancer.

According to an embodiment of the present invention, the effective amount of the lipid nanoparticle is 1×10⁻⁹ M-1×10⁻⁷ M.

Another objective of the present invention is to provide a method for targeting integrin αvβ3, comprising administering to a subject in need thereof a pharmaceutical composition comprising an effective amount of the abovementioned lipid nanoparticle.

Another objective of the present invention is to provide a method for treating a disease related to programmed cell death-ligand 1 (PD-L1), comprising administering to a subject in need thereof a pharmaceutical composition comprising an effective amount of the abovementioned lipid nanoparticle.

Another objective of the present invention is to provide a method for reducing cytotoxicity of an anti-cancer drug to normal cells, comprising administering to a subject in need thereof a pharmaceutical composition comprising an effective amount of the abovementioned lipid nanoparticle.

According to an embodiment of the present invention, the pharmaceutical composition is in a dosage form for intravenous injection.

According to another embodiment of the present invention, the pharmaceutical composition can be further developed into dosage forms such as aerosols, sublingual tablets or patches.

The pH value of normal tissue cells is about 7.2-7.4, while the pH value of tumor tissue cells is about 6-6.5. According to an embodiment of the present invention, a component of the lipid of the lipid nanoparticle is altered so that the lipid nanoparticle triggers a phase transition in a weakly acidic environment, thereby inducing drug release.

According to an embodiment of the present invention, the weakly acidic environment is pH 6-6.5.

In summary, the lipid nanoparticle of the present invention has the following effect. The present invention provides that targeting cell surface integrin αvβ3 nano-liposome specific against digestion system-related cancers locally and systemically. Because integrin αvβ3 is overexpressed on cancer cells or highly growing endothelial cells, even normal cells contain integrin αvβ3, however, the lipid nanoparticle of the present invention only recognizes the conformation of integrin αvβ3 on the cancer cells. Therefore, the lipid nanoparticle of the present invention will target cancer cells instead of normal cells. The lipid nanoparticle of the present invention can equip with other targeting molecules and payload with other anti-cancer drugs. The lipid nanoparticle of the present invention can combine with radiation therapy and reduce the radiation therapeutic threshold (see Cell Cycle. 2011 Jan. 15; 10(2):352-7.; Horm Cancer. 2018 June; 9(3):139-143.)

Embodiments of the present invention will be further described below, the following examples are used to illustrate the present invention, not to limit the scope of the present invention, any person who is familiar with this technique, without departing from the spirit of the present invention and within the scope, some changes and modifications can be made, so the protection scope of the present invention should be determined by the appended claims.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows construction of the lipid nanoparticle of the present invention, in which HSPC represents hydrogenated soy phosphatidylcholine; DSPE represents 1,2-distearoyl-sn-glycero-3-phosphoethanolamine; PEG-DSPE represents polyethylene glycol-1,2-distearoyl-sn-glycero-3-phosphoethanolamine; Dox represents doxorubicin; Tetrac represents tetraiodothyroacetic acid; Lipo-Dox is liposome loaded with doxorubicin; DL-N2 represents the lipid nanoparticle without doxorubicin; DL-N2-Dox represents the lipid nanoparticle loaded with doxorubicin.

FIGS. 2A and 2B show that the lipid nanoparticle (DL-N2) is not cytotoxic to normal cells (HUVEC and Vero cells), but Lipo-Dox and DL-N2-Dox can cause cytotoxicity to normal cells. DL-N2-Dox is induced by doxorubicin (Dox) to normal cells (Vero cells), in which human umbilical vein endothelial cells (HUVEC, ATCC CRL-1730™) and monkey kidney epithelial cells (Vero, ATCC CCL-81™) are treated with tetraiodothyroacetic acid (Tetrac; T3787 Sigma-Aldrich, Burlington, MA) or the lipid nanoparticle (DL-N2) of the present invention for 3 days, and the cell viability is detected, showing that tetraiodothyronacetic acid and DL-N2 do not affect the growth of normal cells (FIG. 2A); monkey kidney epithelial Vero cells (ATCC CCL-81™) are treated with liposomes loaded with doxorubicin (Lipo-Dox), the lipid nanoparticle without doxorubicin (DL-N2) or loaded with doxorubicin (DL-N2-Dox) for 3 days, showing that the cytotoxicity caused by DL-N2-Dox comes from doxorubicin itself (FIG. 2B). Dox represents doxorubicin; Tetrac represents tetraiodothyroacetic acid.

FIGS. 3A and 3B show the cytotoxic effect of lipid nanoparticle on colorectal cancer cells, in which Ras-mutant colorectal cancer HCT116 cells [ATCC® CCL-247™] (FIG. 3A) and Ras-wild type colorectal cancer HT29 cells [ATCC® HTB-38™] (FIG. 3B) are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of colorectal cancer cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; ***p<0.001 as compared with control, #p<0.05 as compared with Lipo-Dox group at the same concentration.

FIGS. 4A and 4B show the cytotoxic effect of the lipid nanoparticle on cholangiocarcinoma cells, in which cholangiocarcinoma HuCC-T1 cells [RCB1960, RIKEN Bioresource Center, Ibaraki, Japan] (FIG. 4A) and cholangiocarcinoma SSP25 cells [RCB1293, RIKEN Bioresource Center, Ibaraki, Japan] (FIG. 4B) are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of cholangiocarcinoma cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01; ###p<0.001 as compared with Lipo-Dox group at the same concentration.

FIG. 5 shows that the lipid nanoparticle inhibits the growth of pancreatic cancer cells, in which pancreatic cancer cells (PANC-1 cells) [ATCC® CRL-1469™] are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of pancreatic cancer cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01 as compared with Lipo-Dox group at the same concentration.

FIGS. 6A and 6B show that the lipid nanoparticle loaded with doxorubicin (DL-N2-Dox) has more inhibitory effect on breast cancer cell growth than Lipo-Dox, in which breast cancer MCF-7 cells [ATCC® HTB-22™] (FIG. 6A) and triple negative breast cancer MDA-MB-231 cells [ATCC® HTB-26™] (FIG. 6B) are treated with different concentrations of liposomes loaded with doxorubicin (Lipo-Dox) or the lipid nanoparticle loaded with doxorubicin (DL-N2-Dox) for 3 days, and the cell viability is detected, showing that the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01; ###p<0.001 as compared with Lipo-Dox group at the same concentration.

FIGS. 7A to 7C show that the antiproliferative effect of the lipid nanoparticle loaded with doxorubicin (DL-N2-Dox) on different types of cancer cells is more potent than that of liposomes loaded with doxorubicin (Lipo-Dox), in which lung cancer A549 cells [ATCC® CCL-185™] (FIG. 7A), cholangiocarcinoma SSP25 cells [RCB1293, RIKEN Bioresource Center, Ibaraki, Japan] (FIG. 7B), and human glioblastoma U87MG cells [ATCC® HTB-14™] (FIG. 7C) are treated with NDAT (10⁻⁷ M) and different concentrations of Lipo-Dox or DL-N2-Dox for 3 days, and the cell viability is detected; data are presented as mean±standard deviation; *p<0.05; **p<0.01; ***p<0.001 as compared with untreated control; #p<0.05; ##p<0.01; ###p<0.001 as compared with Lipo-Dox group at the same concentration.

FIG. 8 shows the effect of the lipid nanoparticle (DL-N2) in inhibiting integrin αv expression in cholangiocarcinoma cells HuCC-T1; ***p<0.001 as compared with untreated control.

FIG. 9 shows the efficacy of the lipid nanoparticle (DL-N2) in inhibiting the expression of programmed cell death 1 ligand 1 (PD-L1) in cholangiocarcinoma HuCC-T1 cells; *p<0.05; ***p<0.001 as compared with untreated control (dimethyl sulfoxide, DMSO); Tetrac represents tetraiodothyroacetic acid; DL-N2 represents the lipid nanoparticle.

FIG. 10 shows the efficacy of the lipid nanoparticle (DL-N2) in inhibiting the expression of epidermal growth factor receptor (EGFR) in cholangiocarcinoma HuCC-T1 cells; *p<0.05; **p<0.01; ***p<0.001 as compared with untreated control (dimethyl sulfoxide, DMSO); Tetrac represents tetraiodothyroacetic acid; DL-N2 represents the lipid nanoparticle.

FIG. 11 shows the efficacy of the lipid nanoparticle (DL-N2) in inhibiting the expression of carcinoembryonic antigen cell adhesion molecule 6 (CECAM6) in cholangiocarcinoma HuCC-T1 cells; *p<0.05; **p<0.01; ***p<0.001 as compared with untreated control (dimethyl sulfoxide, DMSO); Tetrac represents tetraiodothyroacetic acid; DL-N2 represents the lipid nanoparticle.

FIG. 12 shows the effect of the lipid nanoparticle loaded with doxorubicin (DL-N2-Dox) and cisplatin on expression of cyclin D1 (CCND1) and matrix metallopeptidase 9 (MMP9) in cholangiocarcinoma SSP-25 [RCB1293, RIKEN Bioresource Center, Ibaraki, Japan] cells; **p<0.01; ***p<0.001 as compared with control; #p<0.05 as compared with samples treated with cisplatin; DL-N2-Dox represents the lipid nanoparticle loaded with doxorubicin; cisplatin is a platinum-containing anti-cancer drug.

FIG. 13 shows that the lipid nanoparticle of the present invention can extend the life span in mice having tumors, in which immunodeficient mice (NOD SCID, purchased from BioLASCO) were inoculated with Ras-mutant colorectal cancer HCT116 cells [ATCC® CCL-247™] to form tumors, and were injected weekly with tail vein of PBS, DL-N2, Lipo-Dox or DL-N2-Dox drugs once. The result shows that the lipid nanoparticle (DL-N2) of the present invention can prolong the life span of mice compared with the control group (PBS), and DL-N2-Dox can improve the survival rate of mice more than Lipo-Dox.

FIG. 14 shows that tumor location in mice can be targeted by the lipid nanoparticle of the present invention, in which immunodeficient mice (NOD SCID, purchased from BioLASCO) were inoculated with Ras-mutant colorectal cancer HCT116 cells [ATCC® CCL-247™] to form tumors, and were injected with tail vein of PBS, DL-N2, Lipo-Dox or DL-N2-Dox drugs (the drugs carry fluorescent substance Cy7.5 for detection); the distribution of drugs in mice was detected by IVIS live image, and it is found that the lipid nanoparticle of the present invention can target tumor location in mice.

FIG. 15 shows the efficacy of the lipid nanoparticle of the present invention in inhibiting the growth of xenograft tumors, in which immunodeficient mice (Balb/c nude, purchased from BioLASCO) were inoculated with Ras-mutant colorectal cancer HCT116 cells [ATCC® CCL-247™] to form tumors, and were injected weekly with tail vein of PBS, DL-N2, Lipo-Dox or DL-N2-Dox drugs once for 5 weeks. The result shows that the lipid nanoparticle (DL-N2) of the present invention and the lipid nanoparticle loaded with doxorubicin (DL-N2-Dox) can effectively reduce the tumor growth rate. DL-N2 represents the lipid nanoparticle without doxorubicin; DL-N2-Dox represents the lipid nanoparticle loaded with doxorubicin; Lipo-Dox is liposome loaded with doxorubicin, as comparative group; *p<0.05; **p<0.01 as compared with control; #p<0.05 as compared with comparative group.

FIG. 16 shows that the lipid nanoparticle of the present invention can reduce tumor weight, in which immunodeficient mice (Balb/c nude, purchased from BioLASCO) were inoculated with Ras-mutant colorectal cancer HCT116 cells [ATCC® CCL-247™] to form tumors, and were injected weekly with tail vein of PBS, DL-N2, Lipo-Dox or DL-N2-Dox drugs once for 5 weeks. The result shows that the lipid nanoparticle (DL-N2) of the present invention and the lipid nanoparticle loaded with doxorubicin (DL-N2-Dox) can effectively reduce tumor weight. DL-N2 represents the lipid nanoparticle without doxorubicin; DL-N2-Dox represents the lipid nanoparticle loaded with doxorubicin; Lipo-Dox is liposome loaded with doxorubicin, as comparative group; *p<0.05 as compared with control.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

As used herein, the term “treating” or “treatment” refers to alleviating, reducing, ameliorating, relieving, or controlling one or more clinical signs of a disease or disorder, and lowering, stopping, or reversing the progression of severity regarding the condition or symptom being treated.

According to the present invention, the pharmaceutical composition can be manufactured to a dosage form suitable for intravenous injection, using techniques well known to those skilled in the art, or can also be prepared into other dosage forms for administration, including, but not limited to, oral administration, injection (e.g., sterile aqueous solution or dispersion), sterile powder, tablet, troche, lozenge, pill, capsule, dispersible powder or granule, solution, suspension, elixir, slurry, and the like.

According to the present invention, the pharmaceutical composition may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of professionalism and routine techniques of those who are familiar with this technology.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

The present invention is further illustrated by the following examples. The embodiments are provided for illustration only, but not for limiting the scope of the present invention. The scope of the present invention is shown in the appended claims.

### Example 1

### Construction of Lipid Nanoparticle

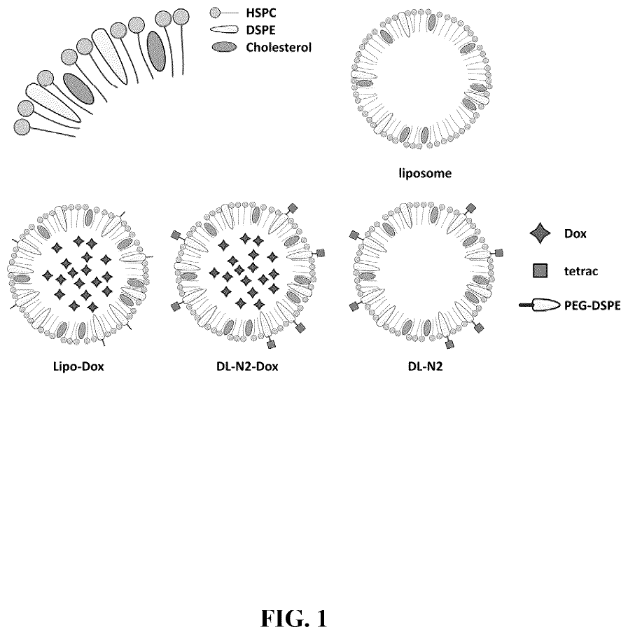

The construction procedure of the lipid nanoparticle of the present invention is as follows. FIG. 1 shows a schematic diagram of the lipid nanoparticle of the present invention. The lipid nanoparticle comprises a hollow lipid sphere and tetraiodothyroacetic acid (Tetrac) covalently linked to the hollow lipid sphere, wherein the hollow lipid sphere is composed of a lipid, and the lipid comprises a hydrogenated soy phosphatidylcholine (HSPC), a cholesterol and 1,2-distearoyl-sn-glycero-3-phosphoethanolamine-polyethylene glycol (DSPE-PEG), wherein the DSPE-PEG is covalently linked to the Tetrac. The construction procedure is as follows. HSPC, cholesterol and DSPE-PEG were mixed in a fixed ratio (the ratio of HSPC, cholesterol and DSPE-PEG is 10-50:1-20:1) and dissolved in absolute alcohol, and then the dissolved lipid solution was quickly injected into the stirring ammonium sulfate aqueous solution with a syringe to form lipid particles. Using high pressure, the lipid particles were gradually shaped into liposomes with a diameter of less than 100 nm through an extruder. The liposomes were replaced with the desired preservation solution and subsequent modifications were performed. Doxorubicin was mixed with liposomes and heated in a water bath at 65° C. for 20 minutes, and cooled down to 4° C. Due to the charge of doxorubicin (Dox), doxorubicin enters liposomes and forms crystals inside, which is Lipo-Dox. The tetraiodothyroacetic acid is bonded with 1,2-distearoyl-sn-glycero-3-phosphoethanolamine-polyethylene glycol (DSPE-PEG) and inserted into the liposomes, so that the tetraiodothyroacetic acid is on the surface of the liposomes, which is the lipid nanoparticle (DL-N2) of the present invention. At the same time, tetraiodothyroacetic acid is bonded with 1,2-distearoyl-sn-glycero-3-phosphoethanolamine-polyethylene glycol (DSPE-PEG) and inserted into Lipo-Dox, so that the tetraiodothyroacetic acid is on the surface of the liposomes, which is the lipid nanoparticle loaded with doxorubicin (DL-N2-Dox) of the present invention.

The pH value of normal tissue cells is about 7.2-7.4, while the pH value of tumor tissue cells is about 6-6.5. According to an embodiment of the present invention, the lipid nanoparticle triggers a phase transition in a weakly acidic environment, thereby inducing drug release.

### Example 2

### Effect of Lipid Nanoparticle on Growth of Normal Cells

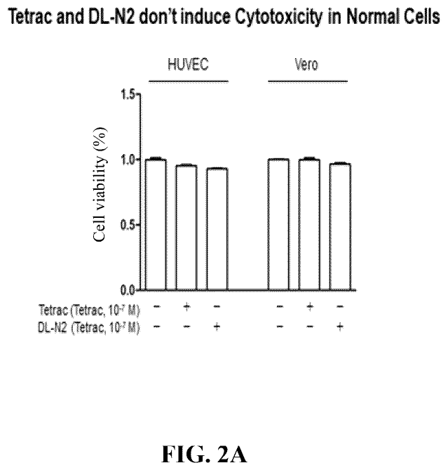

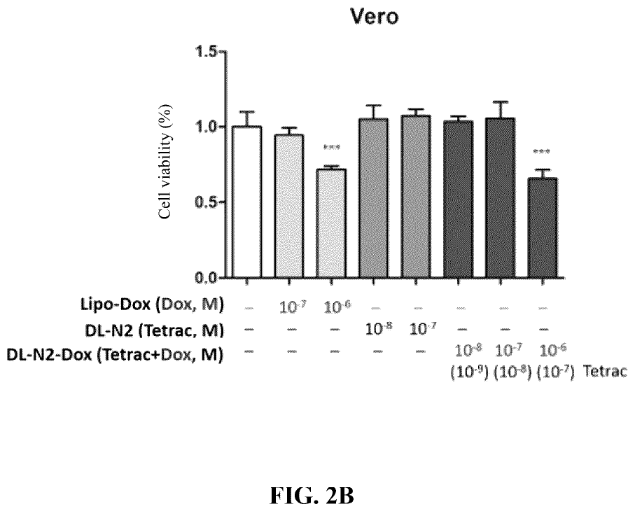

In this example, the effect of the lipid nanoparticle on the growth of normal cells is investigated. The result is shown in FIGS. 2A and 2B.

FIGS. 2A and 2B show that the lipid nanoparticle (DL-N2) is not cytotoxic to normal cells (HUVEC and Vero cells), but Lipo-Dox and DL-N2-Dox can cause cytotoxicity to normal cells. DL-N2-Dox is induced by doxorubicin (Dox) to normal cells (Vero cells), in which human umbilical vein endothelial cells (HUVEC, ATCC CRL-1730™) and monkey kidney epithelial cells (Vero, ATCC CCL-81™) are treated with tetraiodothyroacetic acid (Tetrac; T3787 Sigma-Aldrich, Burlington, MA) or the lipid nanoparticle (DL-N2) of the present invention for 3 days, and the cell viability is detected, showing that tetraiodothyronacetic acid and DL-N2 do not affect the growth of normal cells (FIG. 2A); monkey kidney epithelial Vero cells (ATCC CCL-81™) are treated with liposomes loaded with doxorubicin (Lipo-Dox), the lipid nanoparticle without doxorubicin (DL-N2) or loaded with doxorubicin (DL-N2-Dox) for 3 days, showing that the cytotoxicity caused by DL-N2-Dox comes from doxorubicin itself (FIG. 2B). Dox represents doxorubicin; Tetrac represents tetraiodothyroacetic acid.

From the result of FIGS. 2A and 2B, it can be seen that the liposome loaded with doxorubicin (LipoDox) would negatively affect the cell viability of normal cells, and the lipid nanoparticle without doxorubicin (DL-N2) would not negatively affect the cell viability of normal cells. The lipid nanoparticle loaded with doxorubicin (DL-N2-Dox) only at 10⁻⁶ M doxorubicin and 10⁻⁷ M Tetrac (i.e., DL-N2-Dox (10⁻⁶ M Dox+10⁻⁷ M Tetrac) group) would negatively affect the cell viability of normal cells, showing that the toxic effect of DL-N2-Dox on normal cells comes from doxorubicin (Dox).

### Example 3

### Evaluation of Effect of Lipid Nanoparticle on Inducing Cytotoxicity in Colorectal Cancer Cells

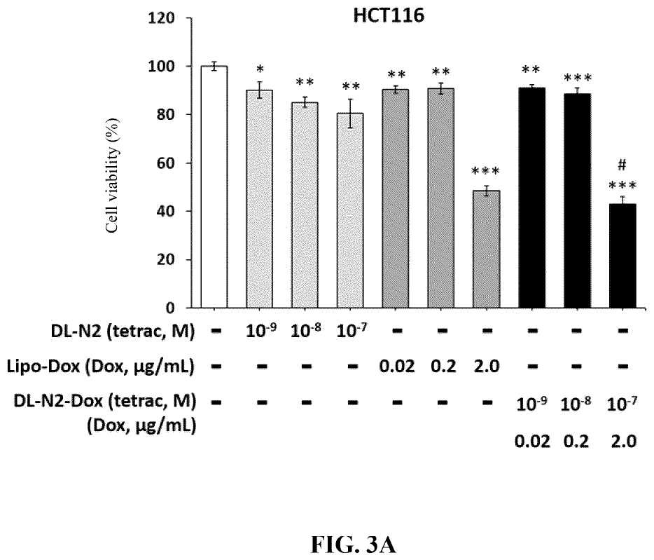

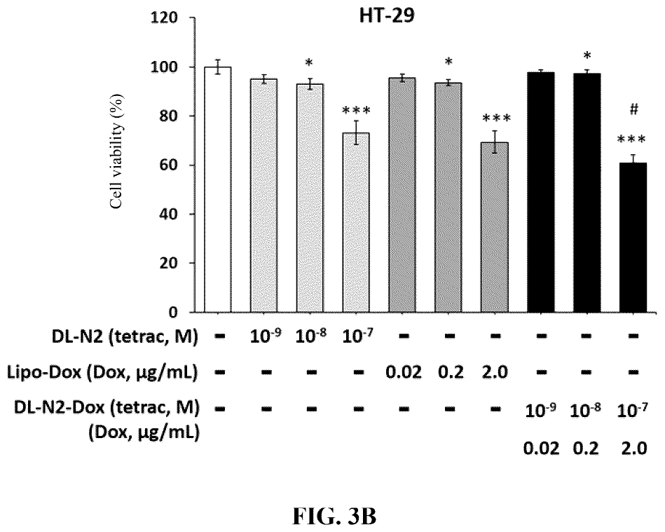

In this example, the effect of the lipid nanoparticle on the growth of colorectal cancer cells is investigated. The result is shown in FIGS. 3A and 3B.

FIGS. 3A and 3B show the cytotoxic effect of lipid nanoparticle on colorectal cancer cells, in which Ras-mutant colorectal cancer HCT116 cells [ATCC® CCL-247™] (FIG. 3A) and Ras-wild type colorectal cancer HT29 cells [ATCC® HTB-38™] (FIG. 3B) are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of colorectal cancer cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; ***p<0.001 as compared with control, #p<0.05 as compared with Lipo-Dox group at the same concentration.

From the result of FIGS. 3A and 3B, the lipid nanoparticle (DL-N2) can be cytotoxic to colorectal cancer HCT116 and HT29 cells in a dose-dependent manner, and the dose of 10⁻⁷ M is more obvious. Therefore, the lipid nanoparticle (DL-N2) can inhibit the proliferation of colorectal cancer cells, and the effect of DL-N2-Dox is better than that of Lipo-Dox at the same concentration.

### Example 4

### Evaluation of Effect of Cytotoxicity of Lipid Nanoparticle on Cholangiocarcinoma Cells

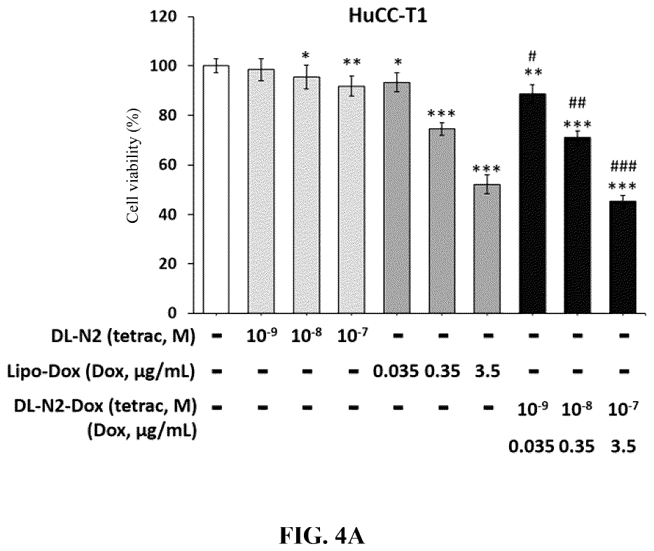

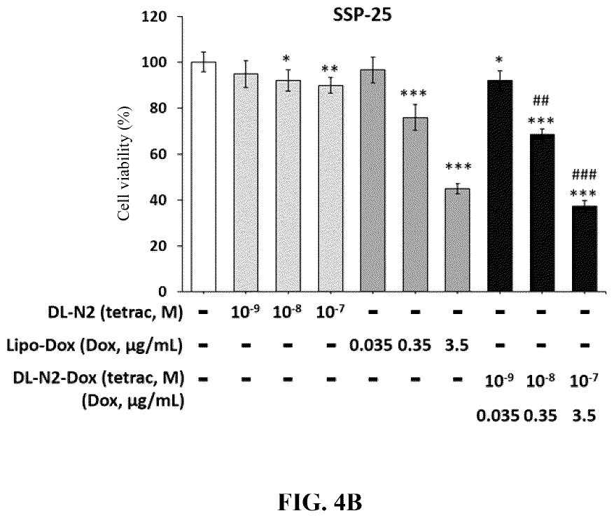

FIGS. 4A and 4B show the cytotoxic effect of the lipid nanoparticle on cholangiocarcinoma cells, in which cholangiocarcinoma HuCC-T1 cells [RCB1960, RIKEN Bioresource Center, Ibaraki, Japan] (FIG. 4A) and cholangiocarcinoma SSP25 cells [RCB1293, RIKEN Bioresource Center, Ibaraki, Japan] (FIG. 4B) are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of cholangiocarcinoma cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01; ###p<0.001 as compared with Lipo-Dox group at the same concentration.

From the result of FIGS. 4A and 4B, the lipid nanoparticle (DL-N2) can inhibit the proliferation of cholangiocarcinoma HCC-T1 and SSP25 cells in a dose-dependent manner, and the dose of 10⁻⁷ M is more obvious. Therefore, the lipid nanoparticle (DL-N2) can inhibit the proliferation of cholangiocarcinoma cells, and the effect of DL-N2-Dox is better than that of Lipo-Dox at the same concentration.

### Example 5

### Evaluation of Lipid Nanoparticle to Inhibit Growth of Pancreatic Cancer Cells

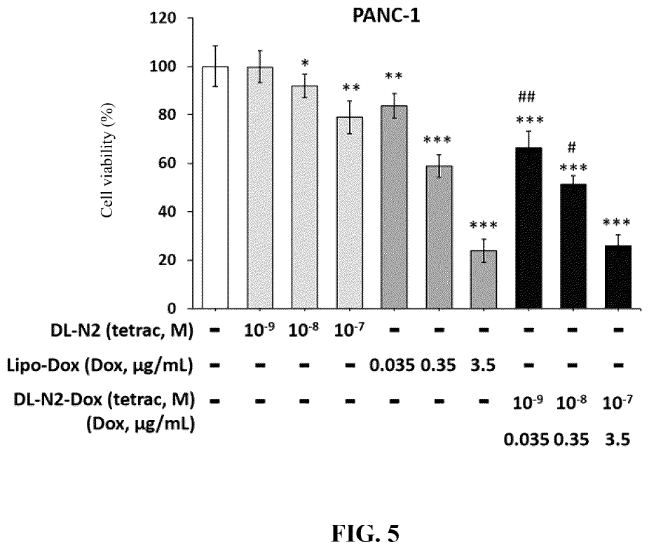

FIG. 5 shows that the lipid nanoparticle inhibits the growth of pancreatic cancer cells, in which pancreatic cancer cells (PANC-1 cells) [ATCC® CRL-1469™] are treated with different concentrations of the lipid nanoparticle (DL-N2), liposomes loaded with doxorubicin (Lipo-Dox) or the lipid nanoparticle loaded with doxorubicin (DL-N2-Dox) for 3 days, and the cell viability is detected, showing that DL-N2 can reduce the cell viability of pancreatic cancer cells and the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01 as compared with Lipo-Dox group at the same concentration.

From the result of FIG. 5, the lipid nanoparticle (DL-N2) can inhibit the growth of pancreatic cancer PANC-1 cells in a dose-dependent manner, and the dose of 10⁻⁷ M is more obvious. Therefore, the lipid nanoparticle (DL-N2) can inhibit the proliferation of pancreatic cancer cells, and the effect of DL-N2-Dox is better than that of Lipo-Dox at the same concentration.

### Example 6

Evaluation of Cytotoxicity of Lipid Nanoparticle Loaded with Doxorubicin (DL-N2-Dox) on Different Types of Breast Cancer Cells

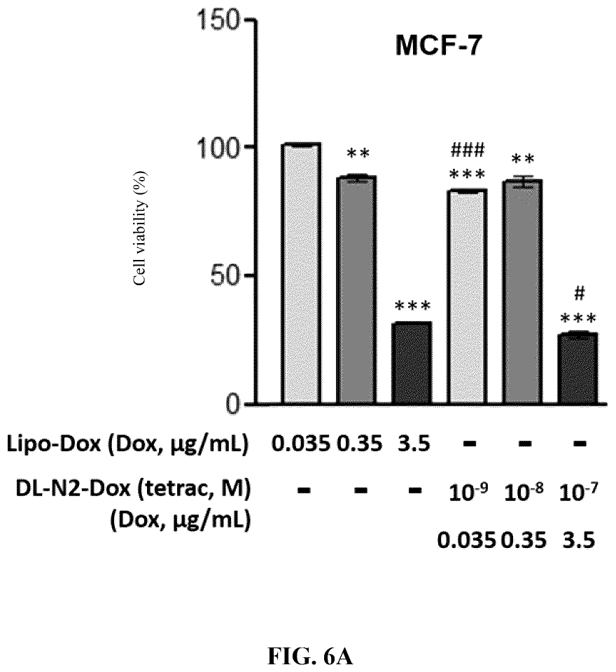

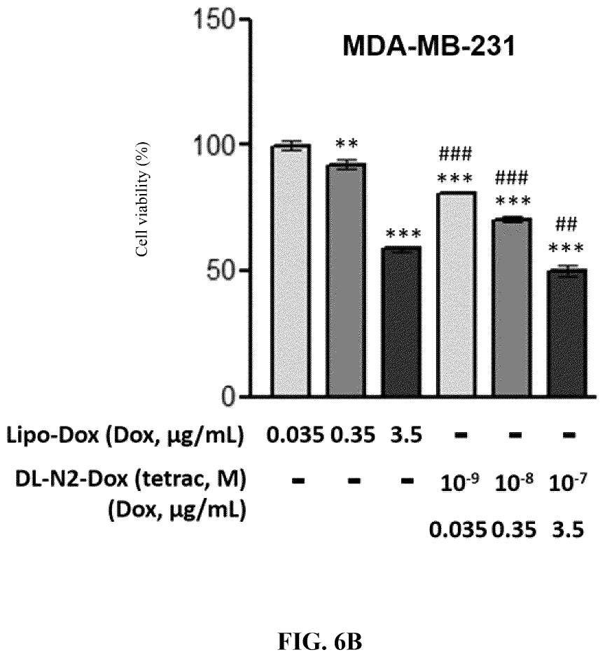

FIGS. 6A and 6B show that the lipid nanoparticle loaded with doxorubicin (DL-N2-Dox) has more inhibitory effect on breast cancer cell growth than Lipo-Dox, in which breast cancer MCF-7 cells [ATCC® HTB-22™] (FIG. 6A) and triple negative breast cancer MDA-MB-231 cells [ATCC® HTB-26™] (FIG. 6B) are treated with different concentrations of liposomes loaded with doxorubicin (Lipo-Dox) or the lipid nanoparticle loaded with doxorubicin (DL-N2-Dox) for 3 days, and the cell viability is detected, showing that the effect of DL-N2-Dox is better than that of Lipo-Dox at the same concentration; Tetrac represents 3,3′,5,5′-Tetraiodothyroacetic acid (T3787 Sigma-Aldrich, Burlington, MA); *p<0.05; **p<0.01; ***p<0.001 as compared with control; #p<0.05; ##p<0.01; ###p<0.001 as compared with Lipo-Dox group at the same concentration.

From the result of FIGS. 6A and 6B, it can be seen that the lipid nanoparticle loaded with doxorubicin (DL-N2-Dox) has inhibitory effects on different types of breast cancer cells, and in triple negative breast cancer MDA-MB-231 cells, the effect of DL-N2-Dox is better than that of Lipo-Dox at the same concentration.

### Example 7

Evaluation of Lipid Nanoparticle Loaded with Doxorubicin (DL-N2-Dox) on Inhibiting Growth of Different Types of Cancer Cells

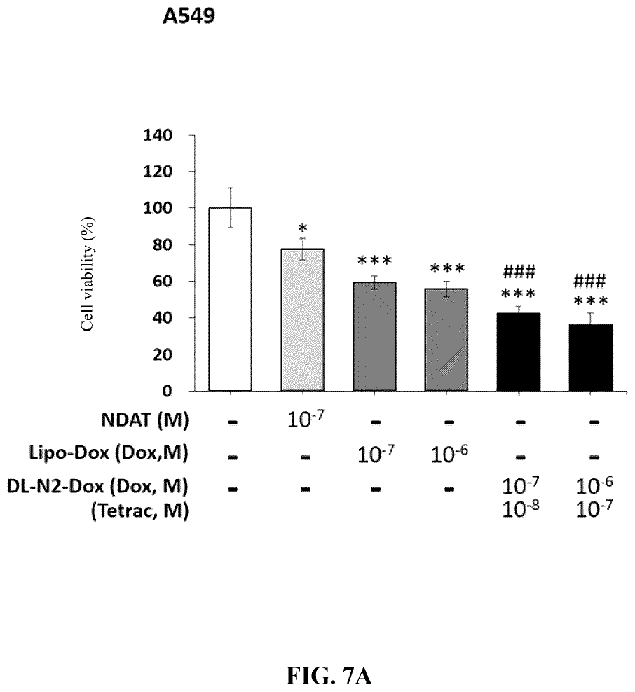

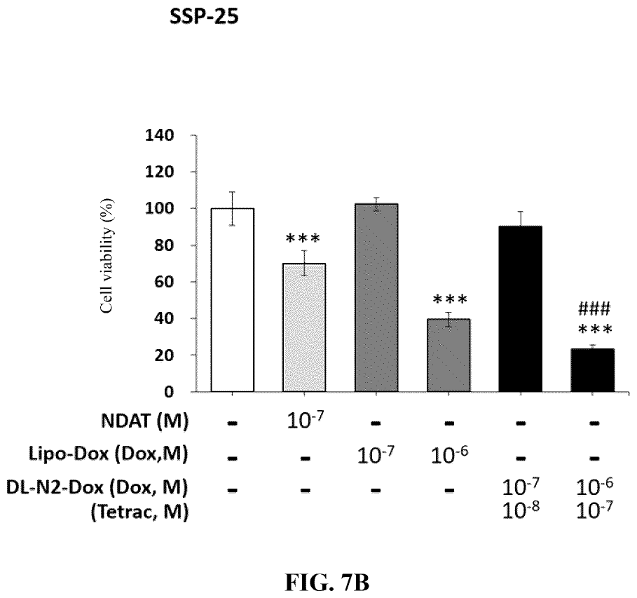

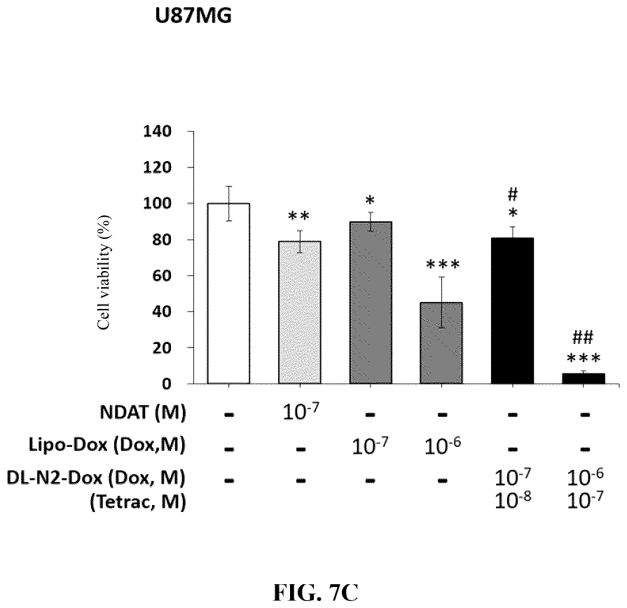

FIGS. 7A to 7C show that the antiproliferative effect of the lipid nanoparticle loaded with doxorubicin (DL-N2-Dox) on different types of cancer cells is more potent than that of liposomes loaded with doxorubicin (Lipo-Dox), in which lung cancer A549 cells [ATCC® CCL-185™] (FIG. 7A), cholangiocarcinoma SSP25 cells [RCB1293, RIKEN Bioresource Center, Ibaraki, Japan] (FIG. 7B), and human glioblastoma U87MG cells [ATCC® HTB-14™] (FIG. 7C) are treated with NDAT (10⁻⁷ M) and different concentrations of Lipo-Dox or DL-N2-Dox for 3 days, and the cell viability is detected; data are presented as mean±standard deviation; *p<0.05; **p<0.01; ***p<0.001 as compared with untreated control; #p<0.05; ##p<0.01; ###p<0.001 as compared with Lipo-Dox group at the same concentration.

From the result of FIGS. 7A to 7C, it can be known that the lipid nanoparticle loaded with doxorubicin (DL-N2-Dox) has more inhibitory effects on lung cancer, cholangiocarcinoma and glioblastoma cells than Lipo-Dox at the same concentration.

### Example 8

### Evaluation of Effect of Lipid Nanoparticle in Inhibiting Integrin αv Expression in Cholangiocarcinoma Cells HuCC-T1

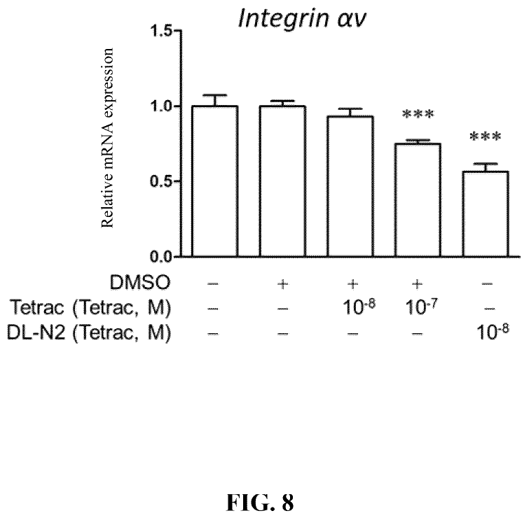

In this example, the effect of the lipid nanoparticle in inhibiting integrin αv expression in cholangiocarcinoma cells HuCC-T1 (RCB1960, RIKEN Bioresource Center, Ibaraki, Japan) is investigated. HuCC-T1 cells were seeded in a 6-well plate and treated with Tetrac (10⁻⁸ or 10⁻⁷ M) or the lipid nanoparticle DL-N2 (10⁻⁸ M) for 24 hours. Cells were harvested and total RNA was extracted. Quantitative polymerase chain reaction (qPCR) experiments were performed to examine the expression of integrin αv. n=4, data are expressed as mean±standard deviation (SD); ***p<0.001 as compared with untreated control. The result is shown in FIG. 8.

Inhibition of integrin αv expression affects cancer metastasis and angiogenesis according to previous studies. It can be seen from the result in FIG. 8 that the lipid nanoparticle of the present invention can inhibit angiogenesis and cancer cell metastasis by inhibiting the expression of integrin αv in cholangiocarcinoma HuCC-T1 cells.

### Example 9

### Evaluation of Efficacy of Lipid Nanoparticle in Inhibiting Expression of Programmed Cell Death 1 Ligand 1 (PD-L1) in Cholangiocarcinoma HuCC-T1 Cells

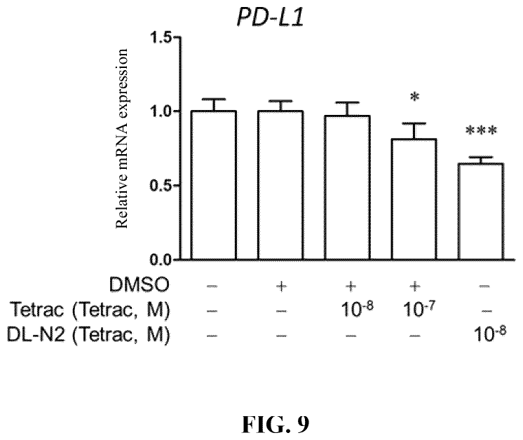

In this example, the efficacy of the lipid nanoparticle in inhibiting the expression of programmed cell death 1 ligand 1 (PD-L1) in cholangiocarcinoma HuCC-T1 [RCB1960, RIKEN Bioresource Center, Ibaraki, Japan] cells is investigated. HuCC-T1 cells were seeded in a 6-well plate and treated with Tetrac (10⁻⁸ or 10⁻⁷ M) or the lipid nanoparticle DL-N2 (10⁻⁸ M or 10⁻⁷ M) for 24 hours. Cells were harvested and total RNA was extracted. Quantitative polymerase chain reaction (qPCR) experiments were performed to examine the expression of PD-L1. n=4, data are expressed as mean±standard deviation (SD); *p<0.05; ***p<0.001 as compared with untreated control (dimethyl sulfoxide, DMSO). The result is shown in FIG. 9.

FIG. 9 shows the efficacy of the lipid nanoparticle (DL-N2) in inhibiting the expression of programmed cell death 1 ligand 1 (PD-L1) in cholangiocarcinoma HuCC-T1 cells; *p<0.05; ***p<0.001 as compared with untreated control (dimethyl sulfoxide, DMSO); Tetrac represents tetraiodothyroacetic acid; DL-N2 represents the lipid nanoparticle. It can be seen from the result in FIG. 9 that the lipid nanoparticle DL-N2 of the present invention can inhibit the expression of PD-L1 in cholangiocarcinoma HuCC-T1 cells.

### Example 10

### Evaluation of Efficacy of Lipid Nanoparticle in Inhibiting Expression of Epidermal Growth Factor Receptor (EGFR) in Cholangiocarcinoma HuCC-T1 Cells

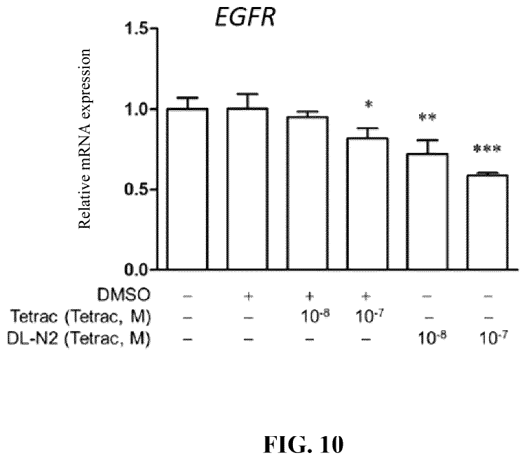

In this example, the efficacy of the lipid nanoparticle DL-N2 in inhibiting the expression of EGFR in cholangiocarcinoma HuCC-T1 [RCB1960, RIKEN Bioresource Center, Ibaraki, Japan] cells is investigated. HuCC-T1 cells were seeded in a 6-well plate and treated with Tetrac (10⁻⁸ or 10⁻⁷ M) or the lipid nanoparticle DL-N2 (10⁻⁸ M or 10⁻⁷ M) for 24 hours. Cells were harvested and total RNA was extracted. Quantitative polymerase chain reaction (qPCR) experiments were performed to examine the expression of EGFR. n=4, data are expressed as mean±standard deviation (SD); *p<0.05; **p<0.01; ***p<0.001 as compared with untreated control (dimethyl sulfoxide, DMSO). The result is shown in FIG. 10.

FIG. 10 shows the efficacy of the lipid nanoparticle (DL-N2) in inhibiting the expression of epidermal growth factor receptor (EGFR) in cholangiocarcinoma HuCC-T1 cells; *p<0.05; **p<0.01; ***p<0.001 as compared with untreated control (dimethyl sulfoxide, DMSO); Tetrac represents tetraiodothyroacetic acid; DL-N2 represents the lipid nanoparticle. It can be seen from the result in FIG. 10 that the lipid nanoparticle can inhibit the expression of EGFR in cholangiocarcinoma HuCC-T1 cells.

### Example 11

### Evaluation of Efficacy of Lipid Nanoparticle in Inhibiting Expression of Carcinoembryonic Antigen Cell Adhesion Molecule 6 (CECAM6) in Cholangiocarcinoma HuCC-T1 Cells

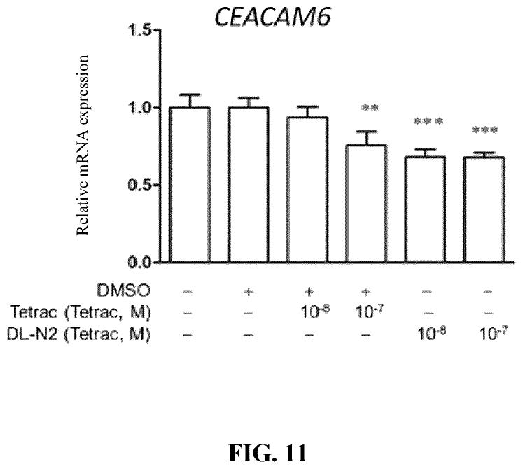

In this example, the efficacy of the lipid nanoparticle in inhibiting the expression of CEACAM6 in cholangiocarcinoma HuCC-T1 [RCB1960, RIKEN Bioresource Center, Ibaraki, Japan] cells is investigated. HuCC-T1 cells were seeded in a 6-well plate and treated with Tetrac (10⁻⁸ or 10⁻⁷ M) or the lipid nanoparticle DL-N2 (10⁻⁸ M or 10⁻⁷ M) for 24 hours. Cells were harvested and total RNA was extracted. Quantitative polymerase chain reaction (qPCR) experiments were performed to examine the expression of CEACAM6. n=4, data are expressed as mean±standard deviation (SD); *p<0.05; **p<0.01; ***p<0.001 as compared with untreated control (dimethyl sulfoxide, DMSO). The result is shown in FIG. 11.

FIG. 11 shows the efficacy of the lipid nanoparticle (DL-N2) in inhibiting the expression of carcinoembryonic antigen cell adhesion molecule 6 (CECAM6) in cholangiocarcinoma HuCC-T1 cells; *p<0.05; **p<0.01; ***p<0.001 as compared with untreated control (dimethyl sulfoxide, DMSO); Tetrac represents tetraiodothyroacetic acid; DL-N2 represents the lipid nanoparticle. It can be seen from the result in FIG. 11 that the lipid nanoparticle can inhibit the expression of CECAM6 in cholangiocarcinoma HuCC-T1 cells.

### Example 12

Effect of Lipid Nanoparticle Loaded with Doxorubicin and Anti-Cancer Drug Cisplatin on Expression of Cyclin D1 (CCND1) and Matrix Metallopeptidase 9 (MMP9) in Cholangiocarcinoma Cells

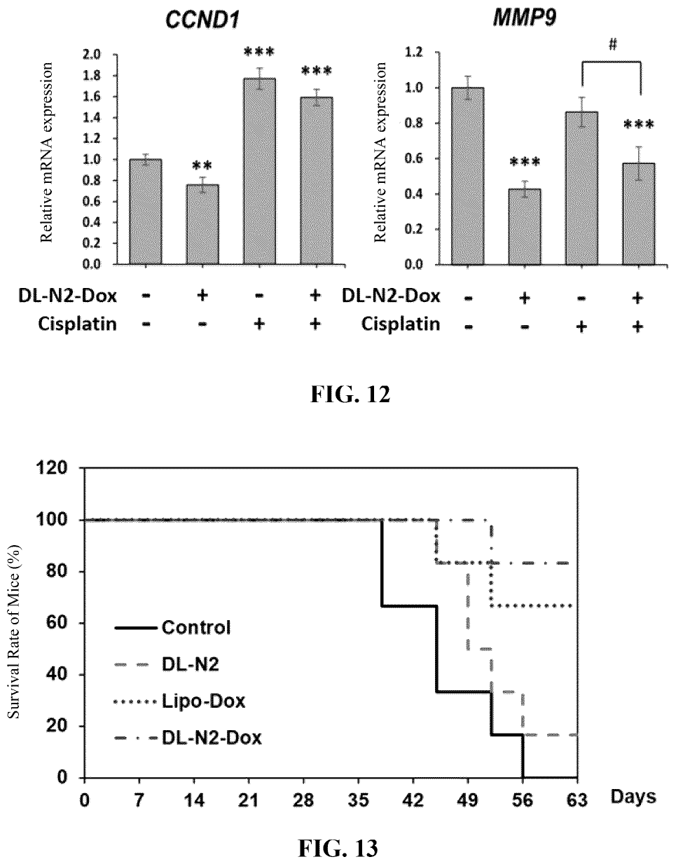

In this example, the effect of the lipid nanoparticle loaded with doxorubicin on the expression of cyclin D1 (CCND1) and matrix metallopeptidase 9 (MMP9) in cholangiocarcinoma SSP-25 [RCB1293, RIKEN Bioresource Center, Ibaraki, Japan] cells is investigated. Cholangiocarcinoma SSP-25 cells were seeded in a 6-well plate and treated with the lipid nanoparticle loaded with doxorubicin (DL-N2-Dox) or cisplatin for 24 hours. Total RNA was extracted and quantitative polymerase chain reaction (qPCR) was performed for CCND1 and MMP9; **p<0.01; ***p<0.001 as compared with control; #p<0.05 as compared with samples treated with cisplatin; cisplatin is a platinum-containing anti-cancer drug. The result is shown in FIG. 12.

From the result of FIG. 12, it can be known that DL-N2 can be used as a carrier to carry other drugs such as doxorubicin, and can also be used in combination therapy to reduce the side effects caused by other drugs such as cisplatin to achieve anti-cancer growth.

### Example 13

### Evaluation of Effect of Lipid Nanoparticle on Extending Life Span in Mice Having Tumors

In this example, the effect of the lipid nanoparticle on extending the life span in mice having tumors is investigated. Immunodeficient mice (NOD SCID, purchased from BioLASCO) were inoculated with Ras-mutant colorectal cancer HCT116 cells [ATCC® CCL-247™] to form tumors, and were injected weekly with tail vein of PBS, DL-N2 (tetrac, 0.1 mg/kg), Lipo-Dox (Dox, 3.5 mg/kg) or DL-N2-Dox (tetrac, 0.1 mg/kg, Dox, 3.5 mg/kg) drugs once. Tumor size was measured twice a week, and for humane consideration, mice with tumors larger than 2 cm³ were sacrificed.

It can be seen from the result in FIG. 13 that the lipid nanoparticle (DL-N2) of the present invention can prolong the life span of mice compared with the control group (PBS), and DL-N2-Dox can improve the survival rate of mice more than Lipo-Dox.

### Example 14

### Lipid Nanoparticle has Ability to Target Tumor Location in Mice

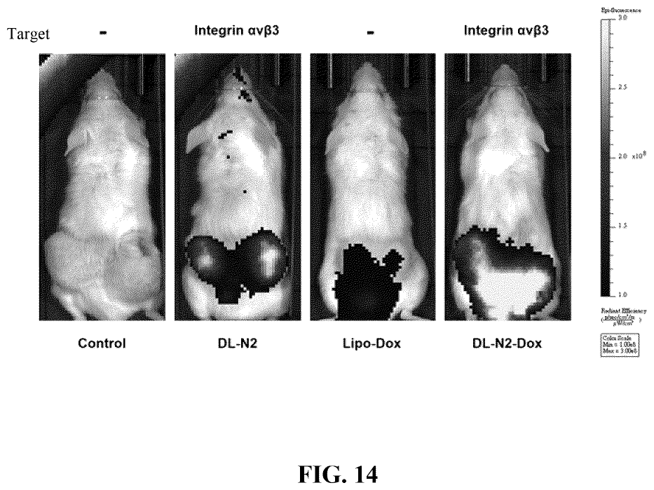

To investigate the ability of the lipid nanoparticle to target tumor integrin αvβ3, immunodeficient mice (NOD SCID, purchased from BioLASCO) were inoculated with Ras-mutant colorectal cancer HCT116 cells [ATCC® CCL-247™] to form tumors, and were injected with tail vein of PBS, DL-N2 carrying fluorescent substance Cy7.5, Lipo-Dox or DL-N2-Dox. The distribution of drugs in mice was detected by IVIS live image. The result is shown in FIG. 14.

It can be seen from the result in FIG. 14 that the lipid nanoparticle of the present invention can target tumor location in mice.

### Example 15

### Evaluation of Efficacy of Lipid Nanoparticle in Inhibiting Growth of Xenograft Tumors

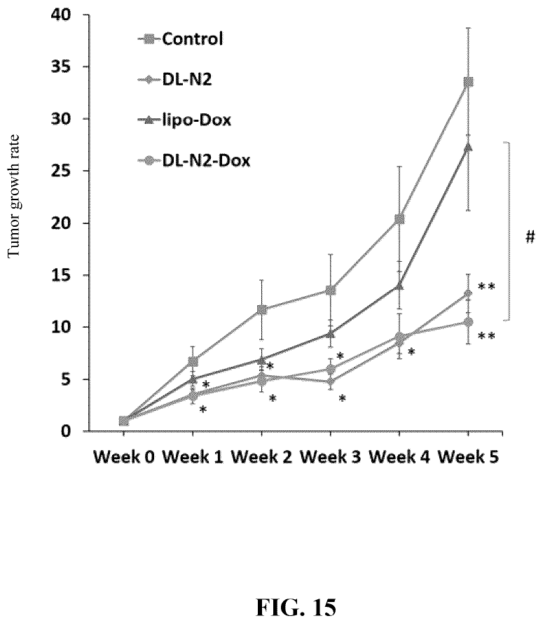

In this example, the efficacy of the lipid nanoparticle in inhibiting growth of xenograft tumors is investigated. Immunodeficient mice (Balb/c nude, purchased from BioLASCO) were inoculated with Ras-mutant colorectal cancer HCT116 cells [ATCC® CCL-247™] to form tumors, and were injected weekly with tail vein of PBS, DL-N2 (tetrac, 0.1 mg/kg), Lipo-Dox (Dox, 2 mg/kg) or DL-N2-Dox (tetrac, 0.1 mg/kg, Dox, 2 mg/kg) drugs once for 5 weeks. The tumor size was measured using a digital caliper, and the volume calculation formula was (length×width×width)×0.53, expressed in cubic millimeters (mm³). The percent increase in tumor volume was calculated by dividing the measured volume on days 0, 7, 14, 21, 28, and 35 by the first measured volume (measured one day before the first drug injection, recorded as day 0). Growth curve of tumor volume normalized to volume before treatment initiation; DL-N2 represents the lipid nanoparticle without doxorubicin; DL-N2-Dox represents the lipid nanoparticle loaded with doxorubicin; Lipo-Dox is liposome loaded with doxorubicin, as comparative group; *p<0.05; **p<0.01 as compared with control (PBS); #p<0.05 as compared with comparative group (lipo-Dox). The result is shown in FIG. 15.

It can be seen from FIG. 15 that both the lipid nanoparticle (DL-N2) of the present invention and the lipid nanoparticle loaded with doxorubicin (DL-N2-Dox) can effectively inhibit the tumor growth of xenograft colorectal cancer, and the effect is better than that of the liposome loaded with doxorubicin (lipo-Dox).

### Example 16

### Evaluation of Lipid Nanoparticle in Inhibiting Xenograft Tumor Weight

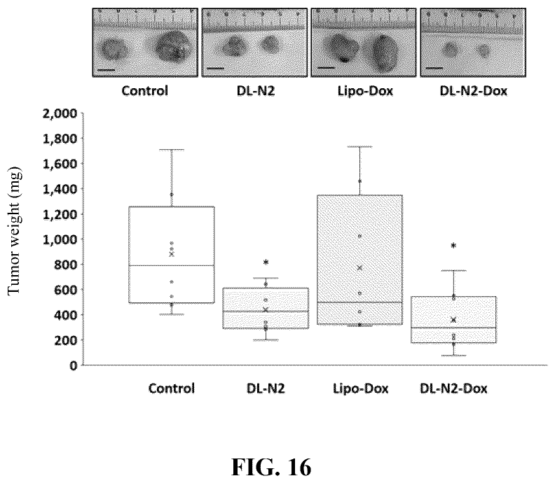

In this example, the effect of the lipid nanoparticle on inhibiting xenograft tumor weight is investigated. Immunodeficient mice (Balb/c nude, purchased from BioLASCO) were inoculated with Ras-mutant colorectal cancer HCT116 cells [ATCC® CCL-247™] on both sides of the back to form tumors, and were injected weekly with tail vein of PBS, DL-N2 (tetrac, 0.1 mg/kg), Lipo-Dox (Dox, 2 mg/kg) or DL-N2-Dox (tetrac, 0.1 mg/kg, Dox, 2 mg/kg) once for 5 weeks. Mice were sacrificed and tumor weight was taken. *p<0.05 as compared with control (PBS). The result is shown in FIG. 16.

It can be seen from FIG. 16 that both the lipid nanoparticle of the present invention (DL-N2) and the lipid nanoparticle loaded with doxorubicin (DL-N2-Dox) can effectively inhibit the tumor size of xenografted colorectal cancer and reduce the tumor weight.

In summary, the lipid nanoparticle of the present invention has the ability to specifically target integrin αvβ3 locally and systemically to cancer cells. Even normal cells contain integrin αvβ3, because integrin αvβ3 is overexpressed on cancer cells or highly growing endothelial cells, the lipid nanoparticle of the present invention only recognizes the conformation of integrin αvβ3 on the cancer cells. Therefore, the lipid nanoparticle of the present invention will target cancer cells instead of normal cells. The lipid nanoparticle of the present invention can equip with other targeting molecules and payload with other anti-cancer drugs. The lipid nanoparticle of the present invention can combine with radiation therapy and reduce the radiation therapeutic threshold.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A lipid nanoparticle, comprising a hollow lipid sphere and tetraiodothyroacetic acid (Tetrac) covalently linked to the hollow lipid sphere, wherein the hollow lipid sphere is composed of a lipid, and the lipid comprises a hydrogenated soy phosphatidylcholine (HSPC), a cholesterol and 1,2-distearoyl-sn-glycero-3-phosphoethanolamine-polyethylene glycol (DSPE-PEG), wherein the DSPE-PEG is covalently linked to the Tetrac.

2. The lipid nanoparticle according to claim 1, further comprising a first drug, wherein the first drug is encapsulated in the hollow lipid sphere.

3. The lipid nanoparticle according to claim 2, wherein the first drug is doxorubicin.

4. The lipid nanoparticle according to claim 2, further comprising a second drug, wherein the second drug is an anti-cancer drug.

5. A method for treating cancer, comprising administering to a subject in need thereof a pharmaceutical composition comprising an effective amount of the lipid nanoparticle according to claim 1.

6. The method according to claim 5, wherein the cancer is colorectal cancer, bile duct cancer, pancreatic cancer, breast cancer or lung cancer.

7. The method according to claim 5, wherein the effective amount of the lipid nanoparticle is 1×10⁻⁹ M-1×10⁻⁷ M.

8. The method according to claim 5, wherein the pharmaceutical composition is in a dosage form for intravenous injection.

9. The method according to claim 5, wherein the lipid nanoparticle triggers a phase transition in a weakly acidic environment, thereby inducing drug release.

10. The method according to claim 9, wherein the weakly acidic environment is pH 6-6.5.

11. A method for targeting integrin αvβ3, comprising administering to a subject in need thereof a pharmaceutical composition comprising an effective amount of the lipid nanoparticle according to claim 1.

12. The method according to claim 11, wherein the pharmaceutical composition is in a dosage form for intravenous injection.

13. The method according to claim 11, wherein a component of the lipid of the lipid nanoparticle is altered so that the lipid nanoparticle triggers a phase transition in a weakly acidic environment, thereby inducing drug release.

14. The method according to claim 13, wherein the weakly acidic environment is pH 6-6.5.

15. A method for treating a disease related to programmed cell death-ligand 1 (PD-L1), comprising administering to a subject in need thereof a pharmaceutical composition comprising an effective amount of the lipid nanoparticle according to claim 1.

16. The method according to claim 15, wherein the pharmaceutical composition is in a dosage form for intravenous injection.

17. The method according to claim 15, wherein a component of the lipid of the lipid nanoparticle is altered so that the lipid nanoparticle triggers a phase transition in a weakly acidic environment, thereby inducing drug release.

18. The method according to claim 17, wherein the weakly acidic environment is pH 6-6.5.

19. A method for reducing cytotoxicity of an anti-cancer drug to normal cells, comprising administering to a subject in need thereof a pharmaceutical composition comprising an effective amount of the lipid nanoparticle according to claim 1.

20. The method according to claim 19, wherein the pharmaceutical composition is in a dosage form for intravenous injection.
